SEQUENCE LISTING (10 sequences)

SEQ ID NO: 1 - DNA, 631 bp, Mus musculus
satgaccgagcgccgcgtgcccttctcgctgctgcggagcccgagctgggaaccattccgggactggtaccctgcacacagccgcctcttcgatcaagctttcggggtgccccggttgcccgatgagtggtcgcagtggttcagcgccgctgggtggcccggatacgtgcgcccgctgcccgccgcgaccgccgagggccccgcggcggtgaccctggccgcaccagccttcagccgagcgctcaaccgacagctcagcagcggggtctcggagatccgacagacggctgatcgctggcgcgtgtccctggacgtcaaccacttcgctccggaggagctcacagtgaagaccaaggaaggcgtggtggagatcactggcaagcacgaagaaaggcaggacgaacatggctacatctctcggtgcttcacccggaaatacacgctccctccaggtgtggaccccaccctagtgtcctcttccctatcccctgagggcacacttaccgtggaggctccgttgcccaaagcagtcacgcagtcagcggagatcaccattccggttactttcgaggcccgcgcccaaattgggggcccagaagctgggaagtctgaacagtctggagccaagtag

SEQ ID NO: 2 - DNA, 618 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaa

SEQ ID NO: 3 - RNA, 23 nt, Artificial Sequence
  Synthetically generated oligonucleotide
nnagcccgagcugggaaccaauu

SEQ ID NO: 4 - DNA, 20 bp, Artificial Sequence
  Synthetically generated oligonucleotide
agcccgagctgggaaccatt

SEQ ID NO: 5 - DNA, 19 bp, Artificial Sequence
  Synthetically generated oligonucleotide
ccgcagagcgtttgagtat

SEQ ID NO: 6 - DNA, 20 bp, Artificial Sequence
  Synthetically generated oligonucleotide
gctcaatccgagagagaata

SEQ ID NO: 7 - DNA, 68 bp, Artificial Sequence
  Synthetically generated oligonucleotide
cgcgtccccagcccgagctgggaaccattcaagagaaatggttcccagctcgggctttttttggaaat

SEQ ID NO: 8 - DNA, 68 bp, Artificial Sequence
  Synthetically generated oligonucleotide
cgcgtccccccgcagagcgtttgagtatttcaagagaatactcaaacgctctgcggttttttggaaat

SEQ ID NO: 9 - DNA, 67 bp, Artificial Sequence
  Synthetically generated oligonucleotide
cgatccccgctcaatccgagaggaatattcaagagatattcctctcggattgagcttttttggaaat

SEQ ID NO: 10 - DNA, 67 bp, Artificial Sequence; identical to SEQ ID NO: 9
  Synthetically generated oligonucleotide

(19) United States
(12) Patent Application Publication
(10) Pub. No.: US 2010/0186102 A1
(43) Pub. Date: Jul. 22, 2010

(54) Methods and compositions for post-transcriptional gene silencing

(75) Inventor: Alexzander Asea, Belton, TX (US)

Correspondence Address: CONLEY ROSE, P.C., 5601 GRANITE PARKWAY, SUITE 750, PLANO, TX, 75024, US

(73) Assignee: Scott and White Memorial Hospital and Scott, Sherwood, and Brindley Foundation, Temple, TX (US)

(21) Appl. No.: 12/673,575

(22) PCT Filed: Aug. 21, 2008
(86) PCT No.: PCT/US08/73872

§ 371 Date: Feb. 15, 2010

Related U.S. Application Data
(60) Provisional application No. 60/957,097, filed on Aug. 21, 2007.

(51) Int. Cl. A01K 67/00; C07H 21/02; C12N 15/63; C12N 5/10; A61K 31/7088; A61K 9/127; A61P 35/04; A61K 39/395; A61K 38/40

(52) U.S. Cl. 800/13; 536/23.1; 536/23.5; 435/320.1; 435/325; 514/44 R; 424/450; 424/138.1; 514/12

(57) ABSTRACT

An isolated double stranded ribonucleic acid (dsRNA) molecule that inhibits the expression of a target gene, the dsRNA comprising two strands of nucleotides wherein a first strand has a length of from 19 to 28 consecutive nucleotides and is substantially identical to a sequence in the target gene and wherein a second strand is substantially complementary to the first strand, and a binding moiety that binds a 3′ end of the first strand to a 5′ end of the second strand. An isolated double stranded ribonucleic acid molecule comprising a first strand of nucleotides that is substantially identical to SEQ ID NO: 3 and a second strand that is substantially complementary to the first.

### STATEMENT REGARDING FEDERALLY SPONSORED RESEARCH OR DEVELOPMENT

Not applicable.

### FIELD

The present disclosure relates to methods and compositions for post-transcriptional gene silencing. More particularly, the disclosure relates to methods and compositions for reducing the expression of heat shock proteins in a cell.

### BACKGROUND

Heat shock proteins (Hsp) are highly conserved proteins found in all prokaryotes and eukaryotes. A wide variety of stressful stimuli, such as for example environmental (U.V. radiation, heat shock, heavy metals and amino acids), pathological (bacterial, parasitic infections or fever, inflammation, malignancy or autoimmunity) or physiological stresses (growth factors, cell differentiation, hormonal stimulation or tissue development), induce a marked increase in intracellular Hsp synthesis which is known as the stress response. This is achieved by activating the trimerization and nuclear translocation of cytoplasmic heat shock factor-1 (HSF-1) to the heat shock element (HSE) within the nucleus and consequent transcription of Hsp. By binding unfolded, misfolded or mutated peptides or proteins and transporting them to the endoplasmic reticulum (ER), Hsp prevents potential aggregation and/or death. Recently, an additional role has been ascribed to Hsp as danger signals produced and released when cells are under stress and as activators of the immune system. The stress response is designed to enhance the ability of the cell to cope with increasing concentrations of unfolded or denatured proteins.

Based on their apparent molecular mass, Hsp are subdivided into two main groups, the small and large Hsp. Hsp25, the murine homologue of human Hsp27, is a ubiquitously expressed member of the small Hsp family that has been implicated in various biological functions. In contrast to large Hsp, Hsp25/27 act through ATP-independent mechanisms and in vivo they act in concert with other chaperones by creating a reservoir of folding intermediates. Hsp25/Hsp27 are associated with estrogen-responsive malignancies and are expressed at high levels in biopsies as well as circulating in the serum of breast cancer patients. Tumor-host interactions play an important role in determining tumor progression, especially in cases that involve metastasis. Biological response modifiers such as Hsp have been shown to orchestrate some of these events. Thus, it would be desirable to develop a composition and method for the regulation of Hsp expression.

### SUMMARY

Disclosed herein is an isolated double stranded ribonucleic acid (dsRNA) molecule that inhibits the expression of a target gene, the dsRNA comprising two strands of nucleotides wherein a first strand has a length of from 19 to 28 consecutive nucleotides and is substantially identical to a sequence in the target gene and wherein a second strand is substantially complementary to the first strand, and a binding moiety that binds a 3′ end of the first strand to a 5′ end of the second strand.

Further disclosed herein is an isolated double stranded ribonucleic acid molecule comprising a first strand of nucleotides that is substantially identical to SEQ ID NO:3 and a second strand that is substantially complementary to the first.

Also disclosed herein is an isolated double stranded ribonucleic acid that inhibits expression of a protein encoded by a nucleic acid molecule comprising a sequence set forth in SEQ ID NO:3; wherein a first strand of the dsRNA is substantially identical to SEQ ID NO:3 and a second strand is substantially complementary to the first.

### BRIEF DESCRIPTION OF THE DRAWINGS

FIG. 1 is an embodiment of a vector.

FIG. 2 is a Western blot of the samples from Example 2.

FIG. 3 is a plot of the number of cells as a function of time for the samples from Example 2.

FIG. 4 is a plot of the tumor volume as a function of time for the samples from Example 2.

FIGS. 5 and 6 are photographs of mice injected with tumor cells and treated as described in Example 2.

FIG. 7 is a plot of the number of invaded cells as a function of the type of shRNA.

FIGS. 8 and 9 are in vivo images of tumor masses treated with shRNAs of this disclosure.

### DETAILED DESCRIPTION

The following are to serve as definitions of terms that may be used throughout this disclosure. A “vector” is a replicon, such as plasmid, phage, viral construct or cosmid, to which another DNA segment may be attached. Vectors are used to transduce and express the DNA segment in cells. As used herein, the terms “vector”, “construct”, “RNAi expression vector” or “RNAi expression construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more RNAi expression cassettes may be or are ligated.

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear or nucleolar RNAs or any kind of RNA transcribed by any class of any RNA polymerase.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell. With respect to eukaryotic cells, a stably transformed cell is one in which the transforming DNA has become integrated into a host cell chromosome or is maintained extra-chromosomally so that the transforming DNA is inherited by daughter cells during cell replication or the transforming DNA is in a non-replicating, differentiated cell in which a persistent episome is present.

Disclosed herein are compositions and methods for selectively reducing the expression of a gene product from a desired targeted gene in a cell or tissue. In an embodiment, the cell is an eukaryotic cell. Also disclosed herein are methods of treating diseases whose course or progression are influenced by the expression of the desired targeted gene. More specifically, disclosed herein are compositions and methods for regulating the expression of heat shock proteins (Hsp). Further disclosed herein are methods for the delivery of compositions that regulate the expression of heat shock proteins to cells and tissues.

In some embodiments, these compositions comprise pharmaceutical formulations comprising therapeutic amounts of materials which may be used in the treatment of an organism experiencing a dysfunction, undesirable medical condition, disorder, or disease state. The dysfunction, undesirable medical condition, disorder, or disease state will be collectively referred to hereinafter as an “undesirable condition.” Herein the undesirable condition is one in which the level of expression of an eukaryotic Hsp may contribute to the onset or progression of the undesirable condition and as such the undesirable condition is one which may be amenable to siRNA therapy. Thus, the undesirable condition includes conditions such as “genetic diseases” which refer to conditions attributable to one or more gene defects, “acquired pathologies” which refer to pathological conditions that are not attributable to inborn defects, cancers, diseases, and the like. Herein “treatment” refers to an intervention performed with the intention of preventing the development or altering the pathology of the undesirable condition. Accordingly “treating” refers both to therapeutic treatments and to prophylactic measures. In an embodiment, administration of therapeutic amounts of compositions of the type described herein to an organism confers a beneficial effect on the recipient in terms of amelioration of the undesirable condition. Herein “therapeutic amounts” refers to the amount of the composition necessary to elicit a beneficial effect. Alternatively, the compositions described herein may be used prophylactically for reducing the potential onset or reoccurrence of an undesirable condition in a recipient not currently experiencing an undesirable condition in which the level of Hsp expression contributes to the onset or reoccurrence of said undesirable condition.

In an embodiment, the compositions comprise one or more isolated or purified nucleic acid molecules (NAMs) and methods of utilizing these NAMs to reduce the expression of one or more Hsp in a cell. As used herein, the term “nucleic acid molecule” (NAMs) can include DNA molecules; RNA molecules; analogs of a DNA or RNA molecule generated using nucleotide analogs; derivatives thereof or combinations thereof. A NAM of the present disclosure can be single-stranded or double-stranded, and the strandedness will depend upon its intended use. Fragments or portions of the disclosed NAMs are also encompassed by the present disclosure. By “fragment” or “portion” is meant less than full length of the nucleotide sequence. As used herein, an “isolated” or “purified” nucleic acid molecule is a nucleic acid molecule that is separated from other nucleic acid molecules that are usually associated with the isolated nucleic acid molecule. Thus, an isolated nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Alternatively, the “isolated” or “purified” NAM may be substantially free of other cellular material or culture medium when produced by recombinant techniques or substantially free of chemical precursors or other chemicals when chemically synthesized. Herein substantially free refers to the level of other components being present in amounts that do not adversely affect the properties of the Hsp reducing compositions and/or the organisms to which the compositions are introduced. For example, the NAMs may be greater than about 70% pure, alternatively greater than about 75%, 80%, 85%, 90%, or 95% pure. Such an isolated nucleic acid molecule is generally introduced into a vector (e.g., a cloning vector, or an expression vector, or an expression construct) for convenience of manipulation or to generate a fusion nucleic acid molecule as will be described in more detail later herein. In addition, an isolated nucleic acid molecule can include an engineered nucleic acid molecule such as a recombinant or a synthetic nucleic acid molecule.

A NAM may be used to regulate the expression of one or more cellular proteins. For example, the NAMs of this disclosure may function to reduce the expression of one or more Hsp. In an embodiment, the NAMs comprise RNA and introduction of the RNA into a cell results in post transcriptional silencing of at least one RNA transcript. The present disclosure provides for such RNA molecules, the DNA molecules encoding such RNA molecules, the polypeptide encoded by such NAMs, antibodies raised to said polypeptides; or combinations thereof. The RNA molecules of this disclosure can be used in a variety of forms; nonlimiting examples of which include antisense RNAi and shRNA.

The disclosed methodologies utilize the RNA interference (RNAi) mechanism to reduce the expression of one or more RNA transcripts. The term “RNA interference or silencing” is broadly defined to include all posttranscriptional and transcriptional mechanisms of RNA mediated inhibition of gene expression, such as those described in P. D. Zamore Science 296, 1265 (2002) which is incorporated by reference herein in its entirety. The discussion that follows focuses on the proposed mechanism of RNA interference mediated by short interfering RNA as is presently known, and is not meant to be limiting and is not an admission of prior art.

RNAi is a conserved biological response that is present in many, if not most, eukaryotic organisms. RNAi results in transcript silencing that is both systemic and heritable, permitting the consequences of altering gene expression to be examined throughout the development and life of an animal.

In the RNAi process, long double-stranded RNA molecules (dsRNA) can induce sequence-specific silencing of gene expression in primitive and multicellular organisms. These long dsRNAs are processed by a ribonuclease called Dicer into 21 to 23 nucleotide (nt) guide RNA duplexes termed short interfering RNA (siRNA). The siRNA is subsequently used by an RNA-induced silencing complex (RISC), a protein-RNA effector nuclease complex that uses siRNA as a template to recognize and cleave RNA targets with similar nucleotide sequences. The composition of RISC is not completely defined, but includes argonaute family proteins. The RISC unwinds siRNAs and associates stably with the (antisense) strand that is complementary to the target mRNA. Depending on the degree of homology between a siRNA and its target mRNA, siRNA-RISC complexes inhibit gene function by two distinct pathways. Most siRNAs pair imperfectly with their targets and silence gene expression by translational repression. This RNAi mechanism appears to operate most efficiently when multiple siRNA-binding sites are present in the 3′-untranslated region of the target mRNAs. In some other cases, siRNAs exhibit perfect sequence identity with the target mRNA and inhibit gene function by triggering mRNA degradation. The reduction in transcript level results in lowered levels of the target protein, resulting in phenotypic changes.

While siRNA has been shown to be effective for short-term gene inhibition in certain transformed mammalian cell lines, there may be drawbacks associated with its use in primary cell cultures or for stable transcript knockdown because their suppressive effects are by definition of limited duration. Short hairpin RNAs (shRNA), consisting of short duplex structures, in contrast to siRNAs have been proved as effective triggers of stable gene silencing in plants, in C. elegans, and in Drosophila. These synthetic forms of RNA may be expressed from pol II or pol III promoters and the hairpin structure is recognized and cleaved by Dicer to form siRNA that is subsequently taken up by RISC for silencing of the target gene.

In an embodiment, the compositions of this disclosure are able to reduce the level of expression of an Hsp, alternatively an eukaryotic Hsp, alternatively a mammalian Hsp. For example, the shRNAs of this disclosure may reduce the expression of a murine Hsp (e.g., Hsp25), a human Hsp (e.g., Hsp27), or both. In an embodiment, the NAMs of this disclosure are able to reduce the expression of polypeptides produced from mRNA transcripts having the sequence set forth in SEQ ID NO:1. Alternatively SEQ ID NO:2.

In some embodiments, the compositions of this disclosure may comprise one NAM that is able to reduce the expression of multiple Hsp. Alternatively, one NAM of the type described herein may exhibit cross reactivity such that it is able to reduce the expression of Hsp from differing species. In either embodiment, the single NAM may inhibit the expression of the differing Hsp to the same extent or to a differing extent. It is also contemplated that the compositions of this disclosure may also reduce the level of expression of one or more Hsp in non-mammalian systems.

The compositions of this disclosure comprise one or more NAMs. In an embodiment, the NAM comprises a double stranded ribonucleic acid (dsRNA) molecule that inhibits the expression of a target gene wherein the dsRNA molecule comprises two strands of nucleotides wherein the first strand is substantially identical to the nucleotide sequence NNAGCCCGAGCUGGGAACCAAUU (SEQ ID NO:3) and wherein the second strand is substantially complementary to the first strand. Herein substantially identical refers to greater than about 50% homology while substantially complementary refers to a complementarity sufficient to permit the annealing of the second strand to the first strand under biological conditions such as within the cytoplasm of a eukaryotic cell.

In an embodiment, the first strand is greater than about 55% homologous, alternatively greater than about 60%, 65%, 70%, 75%, 80%, 90%, 95% homologous to SEQ ID NO:3. The first strand may be of sufficient length such that it is processed by Dicer to produce an siRNA. Either strand may serve as a substrate for Dicer.

The length of each strand generally is from about 19 to about 25 nt in length (e.g., 19, 20, 21, 22, 23, 24, or 25 nucleotides). In some embodiments, the length of each strand is from about 19 to about 28 nucleotides in length. In one embodiment, the length of the sequence in the first strand is identical to the length of the sequence in the second strand and the dsRNA formed is blunt ended. In an alternative embodiment, the ends of the dsRNA formed has overhangs.

In an embodiment, an dsRNA for use in reducing the level of expression of a mammalian Hsp comprises a first strand which includes the sequence 5′-AGCCCGAGCTGGGAACCATT-3′ (SEQ ID NO:4); and/or 5′-CCGCAGAGCGTTTGAGTAT-3′ (SEQ ID NO:5). In an embodiment, a composition for use in the reduction of expression of a Hsp comprises a dsRNA having a first strand which includes the sequence 5′ GCTCAATCCGAGAGAGAATA-3′(SEQ ID NO:6) and a second strand having a sequence complementary to the first strand. In an embodiment, the complementary first and second strands of the dsRNA molecule are the “stem” of a hairpin structure.

The two dsRNA strands can be joined by a binding moiety, which can form the “loop” in the hairpin structure of shRNA. In an embodiment the binding moiety comprises a polynucleotide linker which can vary in length. In some embodiments, the binding moiety can be 5, 6, 7, 8, 9, 10, 11, 12 or 13 nucleotides in length, alternatively the binding moiety is 9 nucleotides in length. A representative binding moiety is 5′-TTC AAG AGA-3′, but any suitable binding moiety that is compatible with the formation of a dsRNA of the type disclosed herein is contemplated. The two strands and binding moiety described herein may form a shRNA that can reduce the expression of one or more Hsp.

NAMs (e.g. dsRNA, shRNA) as described herein can be obtained using techniques known to one of ordinary skill in the art such as for example, recombinant nucleic acid technology; chemical synthesis, either as a single nucleic acid molecule or as a series of oligonucleotides; mutagenesis using common molecular cloning techniques (e.g., site-directed mutagenesis); and the polymerase chain reaction (PCR). General PCR techniques are described, for example in PCR Primer: A Laboratory Manual, Dieffenbach & Dveksler, Eds., Cold Spring Harbor Laboratory Press, 1995 which is incorporated by reference herein in its entirety. Possible mutations include, without limitation, deletions, insertions, substitutions, and combinations thereof. Additionally, suitable molecular biology techniques may be employed for isolation of these molecules such as for example and without limitation restriction enzyme digestion and ligation.

The NAMs disclosed herein may be introduced to a cell directly using techniques such as for example encapsulation in a nanoparticle or a liposome; electroporation; calcium phosphate precipitation and the like. In an embodiment, the NAMs of this disclosure may be introduced to a cell as an element of a vector and thus comprise a DNA vector-based shRNA. Hereinafter, for simplicity the discussion will focus on compositions comprising shRNA although other compositions of the type described previously herein are also contemplated.

Vectors, including expression vectors, suitable for use in the present disclosure are commercially available and/or produced by recombinant DNA technology methods routine in the art. A vector containing a shRNA of this disclosure may have elements necessary for expression operably linked to such a molecule, and further can include sequences such as those encoding a selectable marker (e.g., a sequence encoding antibiotic resistance), and/or those that can be used in purification of a polypeptide (e.g., a His tag). Vectors suitable for use in this disclosure can integrate into the cellular genome or exist extrachromosomally (e.g., an autonomous replicating plasmid with an origin of replication).

In an embodiment, the vector is an expression vector and comprises additional elements that are useful for the expression of the nucleic acid molecules of this disclosure. Elements useful for expression include nucleic acid sequences that direct and regulate expression of nucleic acid coding sequences. One example of an element useful for expression is a promoter sequence. Examples of promoters suitable for use include the mouse U6 RNA promoters, synthetic human H1RNA promoters, SV40, CMV, RSV, RNA polymerase II, RNA polymerase III promoters, derivatives thereof, or combinations thereof. Elements useful for expression also can include ribosome-binding sites, introns, enhancer sequences, response elements, or inducible elements that modulate expression of a nucleic acid. Elements necessary for expression can be of bacterial, yeast, insect, mammalian, or viral origin and the vectors may contain a combination of elements from different origins. Elements necessary for expression are known to one of ordinary skill in the art and are described, for example, in Goeddel, 1990, Gene Expression Technology: Methods in Enzymology, 185, Academic Press, San Diego, Calif., the relevant portions of which are incorporated by reference herein. As used herein, operably linked means that a promoter and/or other regulatory element(s) are positioned in a vector relative to the shRNA in such a way as to direct or regulate expression of the molecule. A shRNA can be operably-linked to regulatory sequences in a sense or antisense orientation. In addition, expression can refer to the transcription of sense mRNA and may also refer to the production of protein.

In an embodiment, the shRNAs of the present disclosure are elements of a retroviral vector. A retroviral vector refers to an artificial DNA construct derived from a retrovirus that may be used to insert sequences into an organism's chromosomes. Adenovirus and a number of retroviruses such as lentivirus and murine stem cell virus (MSCV) are a few of the commonly used retroviral delivery systems. Adenovirus utilizes receptor-mediated infection and does not integrate into the genome for stable silencing experiments, while MSCV cannot integrate into non-dividing cell lines such as neurons, etc. A lentiviral vector is a subclass of retroviral vectors that have the ability to integrate into the genome of non-dividing as well as dividing cells. Lentiviral vectors are known in the art, and are disclosed, for example, in the following publications, which are incorporated herein by reference: Evans J. T. et al. Hum. Gene Ther. 1999; 10:1479-1489; Case S. S., Price, M. A., Jordan C. T. et al. Proc. Natl. Acad. Sci. USA 1999; 96:2988-2993; Uchida N., Sutton R. E., Friera, A. M. et al. Proc. Natl. Acad. Sci. USA 1998; 95:11939-11944; Miyoshi H, Smith K A, Mosier D. E et al. Science 1999; 283:682-686; Sutton R. E., Wu H. T., Rigg R. et al. J. Virol. 1998; 72:5781-5788. The lentiviral vector systems display a broad tropism and non-receptor mediated delivery. Furthermore, lentiviral vector systems have the ability to integrate into the genome for stable gene silencing, without requiring a mitotic event for integration into the genome; thus, extending its use to both dividing and nondividing cell lines. The lentiviral vector system is also not known to elicit immune responses minimizing concerns of off-target effects and use in in vivo applications.

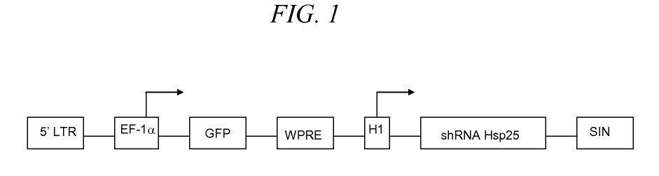

In an embodiment, the shRNAs of the present disclosure are elements of a lentiviral vector. A vector diagram representing an embodiment of a vector suitable for use in this disclosure is shown in FIG. 1. Referring to FIG. 1, features of a typical vector for use in the present disclosure include a promoter such as the elongation factor alpha 1 promoter (EF-1a) disposed upstream of at least one positive selection marker such as the green fluorescent protein (GFP); and one or more regulatory elements such as for example and without limitation the woodchuck hepatitis post-transcriptional regulatory element (WPRE); and at least one NAM sequence for the reduction of Hsp expression (e.g., an shRNA having a first strand comprising SEQ ID NO:4, a complementary second strand and a binding moiety) whose expression may be driven by an upstream polymerase III promoter, human 1 (H1). A regulatory element refers to a genetic element designed to enhance expression of the gene of interest. In one embodiment, the lentiviral vector contains an H1-RNA promoter that is operably linked to a nucleic acid sequence encoding a NAM containing at least one of the sequences previously disclosed herein. Thus, the H1 promoter initiates the transcription of the NAM and allows for the constitutive expression of the NAM. In another embodiment, the NAM is operably linked to a regulatable promoter that provides inducible expression of the NAM. Such inducible promoters and methods of using same are known to one of ordinary skill in the art. In an embodiment, the vector is a lentiviral vector and the markers, genes and other elements of vector may be flanked by an intact retroviral 5′ long terminal repeat (LTR) and 3′ self inactivating (SIN). Such flanking sequences are known to one of ordinary skill in the art.

The types of elements that may be included in the construct are not limited in any way and will be chosen by the skilled practitioner to achieve a particular result. For example, a signal that facilitates nuclear entry of the viral genome in the target cell may be included in the construct. It is to be understood that minor modifications of the vector as disclosed herein may be made without significantly altering the utility of the vector. As such, the vector diagram is not intended to be limiting and is illustrative of one embodiment of a family of vectors. For simplicity hereinafter the family of vectors comprising at least one shRNA as disclosed herein will be referred to as the heat shock protein reduction vector (HRV). In an embodiment, the HRV comprises a lentiviral vector such as for example the LentiGFP Vector commercially available from Lentigen Corp. of Baltimore, Md., the Block-iT Lentivirus Vector commercially available from Invitrogen of Carlsbad, Calif. and the pSIF1-H1 shRNA Vector commercially available from System Biosciences of Mountain View, Calif. and a shRNA of this disclosure.

In an embodiment, the HRV comprises one or more expression cassettes wherein the expression cassette comprises a promoter operably-linked to an isolated nucleic acid sequence encoding a first segment, a second segment located immediately 3′ of the first segment, and a third segment located immediately 3′ of the second segment wherein the first and third segments are from about 19 to about 28 nucleotides in length and wherein the first segment is substantially identical to SEQ ID NO: 3 and wherein the sequence of the third segment is the complement of the first segment. In an embodiment, the isolated nucleated acid sequence expressed from the HRV functions as a shRNA that inhibits the expression of one or more Hsp.

The HRV may be delivered to cells in any way that allows the virus to infect the cell. In an embodiment, the HRV is introduced into a packaging cell line. The packaging cell line provides the viral proteins that are required in trans for the packaging of the viral genomic RNA into viral particles. The packaging cell line may be any cell line that is capable of expressing retroviral proteins. The HRV may then be purified from the packaging cells, titered and diluted to the desired concentration. In one embodiment, the infected cells may be used with or without further processing. In another embodiment, the infected cells may be used to infect an organism.

In an embodiment, the HRV is introduced to a cell or cell line. In another embodiment, the HRV may be introduced to a non-human animal as a genetically modified cell and maintained by the non-human animal in vivo for some period of time. For example, cells may be isolated from the non-human animal and the HRV introduced into cells using any number of in vitro techniques as have been described previously herein (e.g. electroporation, calcium phosphate precipitation, etc.). The isolated cells now carrying the HRV may be reintroduced to the non-human animal and result in the reduced expression of one or more Hsps for some period of time. In other embodiments, similar methodologies may be employed for treating a human having an undesired condition.

In an embodiment, cells, tissue, or an organism having been infected with an HRV as disclosed herein may experience a reduced level of Hsp expression when compared to an otherwise similar cell or organism lacking an HRV. For example, cells expressing a Hsp when infected with an HRV comprising SEQ ID NOS 4, 5, or 6 may experience a reduction in the level of Hsp expression.

The Hsp expression level in a cell or organism comprising an HRV may be reduced by an amount of equal to or greater than about 60%, alternatively greater than about 70, 75, or 80% when compared to an otherwise identical cell or organism in the absence of an HRV. Methods for determining the reduction in the Hsp expression level may comprise assays for the mRNA transcript; assays for the translated product, or combinations thereof. NAMs (e.g., mRNA transcript) and polypeptides (e.g., Hsp) can be detected using a number of different methods well known to one of ordinary skill in the art. Methods for detecting NAMs include, for example, PCR and nucleic acid hybridizations (e.g., Southern blot, Northern blot, or in situ hybridizations).

The shRNAs of the present disclosure can be used to reduce the expression of Hsp in a number of cell types or tissue types. As such the shRNAs may be introduced to any cell type or tissue experiencing an undesirable condition for which reduction of the expression of Hsp may ameliorate said condition. For example, the shRNAs of the present disclosure can be used to reduce the expression of Hsp in cancer cells. As used herein, “cancer cells” refer to cells that grow uncontrollably and/or abnormally, and can be, for example, epithelial carcinomas. Epithelial carcinomas include, for example, head and neck cancer cells, breast cancer cells, prostate cancer cells, and colon cancer cells. The shRNAs of the present disclosure may be administered so as to result in an inhibition of the proliferation of cancer cells. Proliferation of cancer cells as used herein refers to an increase in the number of cancer cells (in vitro or in vivo) over a given period of time (e.g., hours, days, weeks, or months). It is noted that the number of cancer cells is not static and reflects both the number of cells undergoing cell division and the number of cells dying (e.g., by apoptosis). An inhibition of the proliferation of cancer cells can be defined as a decrease in the rate of increase in cancer cell number, a complete loss of cancer cells, or any variation there between. With respect to tumors, a decrease in the size of a tumor can be an indication of an inhibition of proliferation. The administration of one or more compositions comprising an shRNA of the type described herein to an organism having a cell proliferation disorder evinced by tumor growth may result in an inhibition of tumor growth of from about 10% to about 90%, alternatively from about 30% to about 90%, alternatively greater than about 75% when compared to the tumor cell growth observed in the absence of the HRV. Herein the tumor cell growth refers to cell proliferation or increase in tumor mass and may be measured by techniques known to one of ordinary skill in the art such as for example magnetic resonance imaging, electronic caliper, mammogram.

Further, the shRNAs of the present disclosure may result in the cancer having a reduced metastatic potential. Metastasis refers to the spread of cancerous cells from its primary site to other sites in the body. Thus, the shRNAs of this disclosure when introduced and expressed in cancer cells having a metastatic potential may reduce the ability of the cancerous cells to spread from the primary site when compared to the metastatic potential of cells not expressing the shRNAs of this disclosure. The administration of one or more compositions comprising an shRNA of the type described herein to an organism having a cell proliferation disorder evinced by tumor growth with the potential to metastasize may result in reduction in the metastatic potential of from about 10% to about 95%, alternatively from about 30% to about 70%, alternatively equal to or greater than about 75% when compared to the tumor cell growth observed in the absence of the HRV. Herein metastatic potential refers to the ability of the tumor to grow at one more distal sites and may be measured by techniques known to one of ordinary skill in the art such as for example cell migration assays.

In an embodiment, the compositions comprising shRNAs of the type described herein may be used in conjunction with other therapeutic methods to effect the treatment of an undesirable condition. For example, the shRNAs of this disclosure may be used in conjunction with other gene silencing therapies, chemotherapeutic regimes, radiation therapies, hypothermia, and the like.

In an embodiment, the shRNAs of this disclosure may be a component in a pharmaceutical composition wherein the composition is to be administered to an organism experiencing an undesired condition and act as a therapeutic agent. The pharmaceutical composition (PC) may be formulated to be compatible with its intended route of administration. For example, the organism may have one or more tumor loads and the PC may be introduced via direct injection. Additionally, examples of routes of administration include parenteral (e.g., intravenous, intradermal, subcutaneous); oral (e.g., ingestion or inhalation); transdermal (e.g., topical); transmucosal; and rectal administration. In an embodiment, the shRNAs of the present disclosure either alone or as a component of a vector (i.e. HRV) can be incorporated into pharmaceutical compositions suitable for administration. Such compositions typically comprise the shRNAs, and a pharmaceutically acceptable carrier or excipient. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art.

In an embodiment, a composition for use in the treatment of an undesirable condition comprises administration of a tumor targeting Hsp reduction system (TTHRS). The TTHRS may comprise one or more of the Hsp compositions previously described herein, one or more delivery nanoparticles, and one or more targeting moieties. In an embodiment, the TTHRS is capable of delivering the Hsp reducing compositions of this disclosure to tumor cells wherever they may occur in the body. For example, the TTHRS may be capable of delivering the compositions of this disclosure to both primary and metastatic disease.

In an embodiment, the TTHRS comprises a delivery system for the transport of one or more shRNAs and optional components in an organism. Delivery systems may include the use of any materials compatible with the compositions of this disclosure and suitable for use in an organism. In an embodiment, the delivery system comprises a nanoparticle, alternatively a liposome. Herein nanoparticle refers to a material wherein at least one dimension is less than about 100 nm in size while liposome refers to a bilayer lipid. Liposomes generally have systemic applications as they exhibit extended circulation lifetimes following intravenous (i.v.) injection, can accumulate preferentially in various tissues and organs or tumors due to the enhanced vascular permeability in such regions, and can be designed to escape the lyosomic pathway of endocytosis by disruption of endosomal membranes. Liposomes generically comprise an enclosed lipid droplet having a core, typically an aqueous core, containing the compound. The liposomes or liposome precursors may be prepared using any means known to one of ordinary skill in the art. An example of liposomes suitable for use in this disclosure are the DOTAP series of cationic lipids which are substituted N-(1-(2,3-dioleoyloxy)propyl)-N,N,N-trimethylammonium chloride compounds commercially available from Avanti Polar Lipids. In certain embodiments, the Hsp reducing compositions of this disclosure are chemically conjugated to a lipid component of the liposome. In other embodiments, the Hsp reducing compositions of this disclosure are contained within the aqueous compartment inside the liposome.

In an embodiment, the TTHRS comprises a targeting moiety. Such targeting moieties may recognize and bind to receptors on the surface of cells. In an embodiment, the targeting moieties may be chosen so as to preferentially bind receptors that are expressed primarily by a dysfunctional or diseased cell. Alternatively, the diseased cells may express elevated levels of one or more receptors such that while the targeting moiety may bind both normal and diseased cells, the diseased cells will be targeted to a greater extent than a normal cell. In an embodiment, the targeting moieties may comprise any material which is compatible with the other components of the TTHRS and able to bind efficiently to one or more cells of interest (e.g., tumor cells). Such moieties are known in the art and may include antibodies, transferrin, and the like. In an embodiment, the targeting moiety comprises transferrin. In an embodiment, the TTHRS comprises transferrin which is associated with the surface of the liposome of the TTHRS.

Additionally disclosed herein are articles of manufacture (e.g., kits) that contain one or more shRNAs, one or more vectors that encode a shRNA of the present disclosure (e.g. HRV) or one or more TTHRS. Such compositions may be formulated for administration and may be packaged appropriately for the intended route of administration as described previously herein. For example, a shRNA or a vector comprising a shRNA of the present disclosure can be contained within a pharmaceutically acceptable carrier or excipient.

In an embodiment, a kit comprising a shRNA or HRV of the present disclosure also can include additional reagents (e.g., buffers, co-factors, or enzymes). Pharmaceutical compositions as described herein further can include instructions for administering the composition to an individual. The kit also can contain a control sample or a series of control samples that can be assayed and compared to the biological sample. Each component of the kit is usually enclosed within an individual container and all of the various containers are within a single package.

### EXAMPLES

The invention having been generally described, the following examples are given as particular embodiments of the invention and to demonstrate the practice and advantages thereof. It is understood that the examples are given by way of illustration and are not intended to limit the specification of the claims to follow in any manner.

### Example 1

The ability of nucleic acid molecules containing the shRNA sequences given in SEQ ID NO:6 and SEQ ID NO:7 to reduce the expression of Hsp 25 was investigated. Murine breast carcinoma 4T1 cells are a 6-thioguanine-resistant cell line selected from 410.4 tumor without mutagen treatment. The cells were maintained in Dulbecco's modified Eagle medium (Invitrogen, Carlsbad, Calif., USA) containing 2 mM L-glutamine and adjusted to contain 1.5 g/l sodium bicarbonate, 4.5 g/l glucose, 10 mM HEPES, 1.0 mM sodium pyruvate and 10% fetal bovine serum at 37° C. in a humidified incubator with a 5% CO₂ atmosphere. Cells were grown at an exponential growth rate and harvested using 0.1% trypsin-EDTA when cultures are approximately 80% confluent. Cells were passaged only 5-8 times before fresh cells were used.

Two samples containing 4T1 cells were transfected with either vector pLVTHM, psPAX2 or pMD2G each containing shRNA having either SEQ ID NO:8 or SEQ ID NO:9 and GFP^{plasmid} using the lipid transfection reagent Effectene according to the manufacturer's instructions (Qiagen, Valencia, Calif., USA). In the following discussion, SEQ ID NO:6 is referred to as AS1or Hsp25shRNA1 while SEQ ID NO: 7 is referred to as DS1 or Hsp25shRNA2. A third sample was transfected with a control sequence SEQ ID NO:10. CGATCCCCGCTCAATCCGAGAGGAATATTCAAGAGATATTCCTCTCGGATTGAGCTTT TTTGGAAAT. Briefly, 3×10⁵ exponentially growing cells were seeded in 60-mm tissue culture plates and a mixture of 1 μg GFP^{plasmid} DNA and 1 μg of the plasmid containing AS1, DS1 or a control in Effectene was added to the cells and incubated for 18 h at 37° C. After 48 h, cells were harvested and immediately sorted into GFP-positive and -negative subpopulations using a MoFlow cytometer (Dakocytomation, Carpinteria, Calif., USA). Individual cells were gated on the basis of forward scatter (FSC) and orthogonal scatter (SSC). The photomultiplier (PMT) for GFP (FL1-height) was set on a logarithmic scale. Cell debris was excluded by raising the FSC-height PMT threshold. The flow rate was adjusted to ×200 cells/s and at least 10⁵ cells were sorted for each sample group.

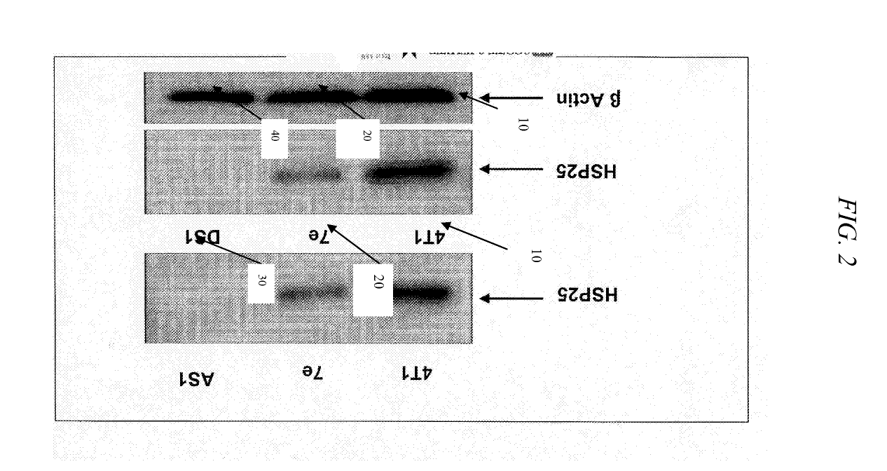

One million cells were lyzed using RIPA buffer containing appropriate protease inhibitors, and the protein concentration was determined using the Bradford method (Bio-Rad, Hercules, Calif., USA) with a DU-650 Spectrophotometer (Beckman Coulter). Samples were run in a 12% SDS-PAGE gel and transferred onto a nitrocellulose membrane. The membrane was blocked for 1 h at 4° C. with Tween 20-Tris-buffered saline (T-TBS) containing 5% milk. After rinsing, the membrane was probed with a primary antibody against Hsp27 (StressGen Biotechnologies) in a dilution ratio of 1:2,000 or Hsp25 (StressGen Biotechnologies) in a dilution ratio of 1:1,000. Antibodies were diluted in T-TBS containing 5% milk. After 1 h of incubation at room temperature, the membrane was washed in T-TBS three times. Corresponding HRP-conjugated IgG secondary antibodies (Sigma-Aldrich, St. Louis, Mo., USA) were added and the membrane was incubated for 30 min at room temperature. After additional washes, bands were visualized using enhanced chemiluminescence (Amersham, Little Chalfont, UK) and the results are shown in FIG. 2.

FIG. 2 shows the blots for cells transfected with AS1, DS1, or a control shRNA. β-actin was used as the loading control. The 4T1 cells, reference arrows 10, are seen to express Hsp-25 in both experiments. Transfection of the cells with a control shRNA (SEQ ID NO:6) results in a reduction in Hsp expression, reference arrows 20, however, there is no detectable expression of Hsp25 in cells transfected with AS1 or DS1, reference arrows 30 and 40 respectively.

### Example 2

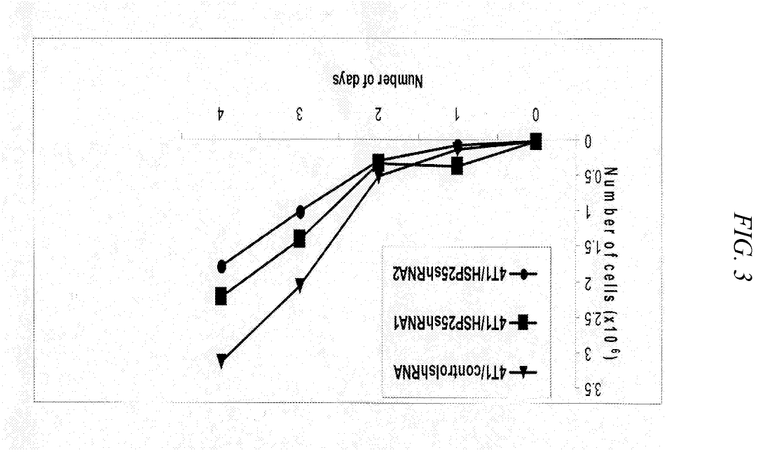

The growth of tumor cells transfected with the vectors described in Example 1 was investigated. The cell growth of 4T1 was measured using a hematocytometer for a total of 4 days and the results of the growth are shown in FIG. 3 where the graph is labeled as follows: control shRNA corresponds to 4T1/controlshRNA1; AS1 corresponds to 4T1/HSP25 shRNA1; and DS1 corresponds to 4T1/HSP25shRNA2. The results demonstrate that cells expressing the control shRNA, AS1, or DS1 displayed similar growth curves.

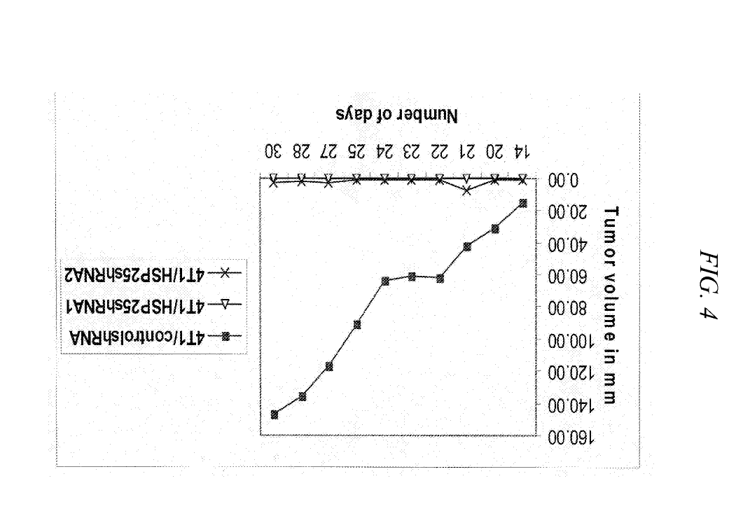

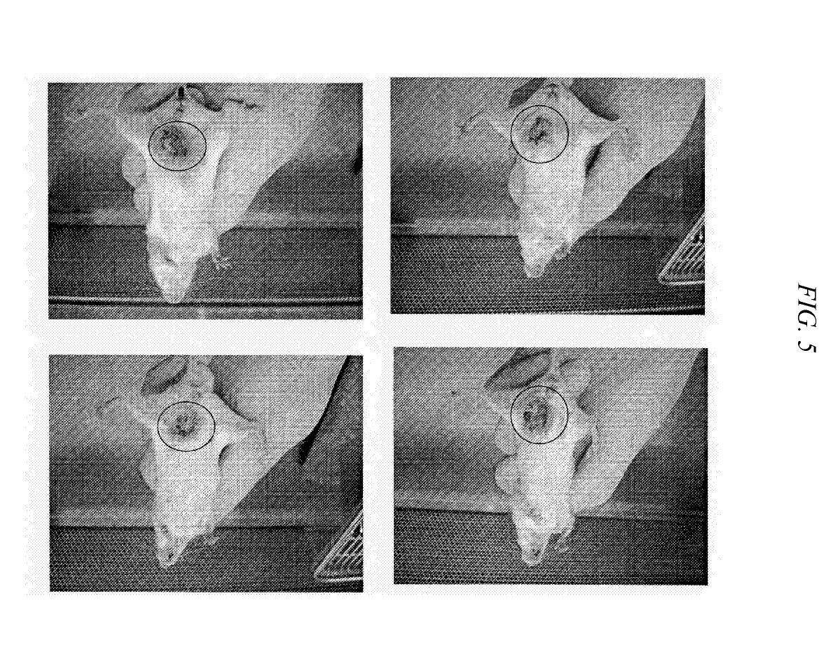

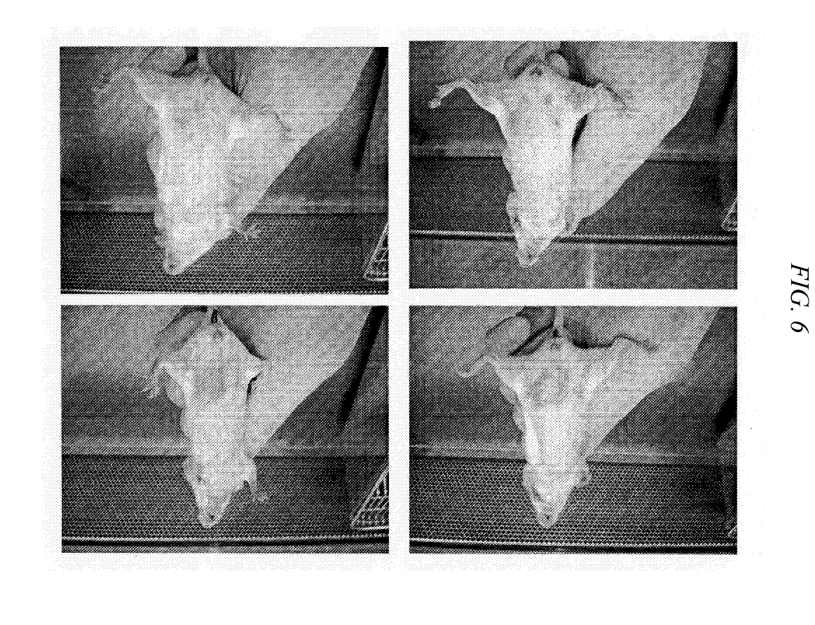

The tumor cells transfected with the vectors described in Example 1 were used to infect animals and primary tumor development in those animals were investigated. Specifically, BALB/c mice purchased from Jackson Laboratories (Bar Harbor, Me., USA) were challenged by injection of 4T1 cells into the abdominal mammary gland, and tumor volume was measured at regular intervals using an electronic caliper until tumor size reached 1,000 mm³. The tumor volume was estimated using the formula for the volume of an ellipsoid (length×width×height×0.5236). All animals were treated humanely and in accordance with the guidelines of the Committee on the Care and Use of Laboratory Animals of the Institute of Animal Resources, National Research Council and Boston University School of Medicine. The primary tumor growth curves for animals infected with cells expressing a control shRNA, AS1, or DS1 are shown in FIG. 4 where the graph is labeled as follows: control shRNA corresponds to 4T1/controlshRNA1; AS1 corresponds to 4T1/HSP25shRNA1; and DS1 corresponds to 4T1/HSP25shRNA2. The results demonstrate that animals injected with tumor cells transfected with AS1 or DS1 showed small changes in tumor volume over the course of the experiment whereas animals injected with tumor cells transfected with a control shRNA had a substantial growth in tumor volume over the course of the experiment. This is further illustrated in FIGS. 5 and 6 which show photographs of mice that had been injected with tumor cells transfected with AS1, FIG. 5 or with tumor cells transfected with a control shRNA, FIG. 6. The mice in FIG. 4 infected with tumor cells transfected with AS1 showed little to no development of a solid tumor over the course of the experiment whereas the mice injected with tumor cells transfected with a control shRNA had tumor development over the course of the experiment.

### Example 3

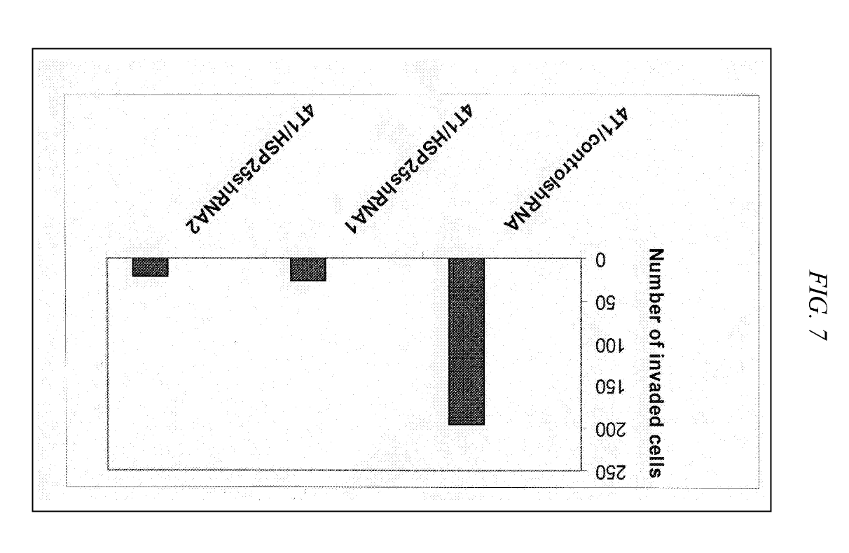

The ability of the AS1 and DS1 molecules described in Example 1 to reduce the metastatic potential of tumor cells was investigated using a cell migration assay. Cell migration was measured using the Matrigel invasion chambers (BD Biocoat Cellware, San Jose, Calif., USA) according to the manufacturer's instructions and 4T1 tumor cells described in Example 1. Briefly, conditioned medium was placed in the lower chamber as a chemoattractant. Single-cell suspensions were placed on the upper chamber. Twenty-two hours later, cells that had not penetrated the filter were washed off and the membrane stained with 0.5% crystal violet, mounted on a microscope slide, visualized and photographed. Fifteen different fields were visualized using a light microscope at 10× magnification. FIG. 7 is a plot of the number of invaded cells for each construct where invasion refers to the number of tumor cells that migrated toward the chemoattractant where the graph is labeled as follows: control shRNA corresponds to 4T 1/controlshRNA1; AS1 corresponds to 4T1/HSP25shRNA1; and DS1 corresponds to 4T1/HSP25shRNA2. The results demonstrate that tumor cells transfected with either the AS1 or DS1 construct migrated to a lesser extent than the tumor cells transfected with the control shRNA.

### Example 4

Briefly, liposomes consisting of DOTAP and Cholesterol (1:1 molar ratio) were prepared by thin film hydration then membrane extrusion to get 80-100 nm particle size as measured using N4 PLUS Coulter particle size scattering instrument. Liposome nanoparticles contained DOTAP/Cholesterol, protamine sulfate and the Hsp targeting siRNA oligonucleotides of the type disclosed in SEQ ID NOs: 4-6 and a control sequence. To prepare 1 mg/kg bodyweight siRNA formulations, 200 μl liposome nanoparticles contains 13.5 μl siRNA, 10 μl (20 μg) protamine sulfate, 40 μl DOTAP and Cholesterol (1:1 molar ratio), 15 μl Transferrin (300 μg), 121.5 μl RNase free water. DOTAP, Cholesterol is commercially available from Avanti Polar Lipids, Inc., human transferrin in the iron-saturated, heat inactivated form is commercially available from BD Biosciences, and protamine sulfate Grade X isolated from salmon is commercially available from Sigma-Aldrich. The nanoparticle complex will be prepared by mixing the protamine sulfate, RNase free water, siRNA and allowed to stand at room temperature for 10 min before the addition of DOTAP/Cholesterol liposome, transferrin complex. The liposome nanoparticles were incubated at room temperature for 10 min before injection into animals.

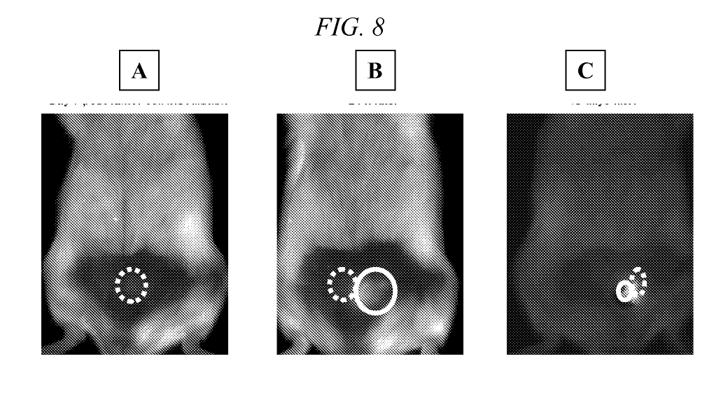

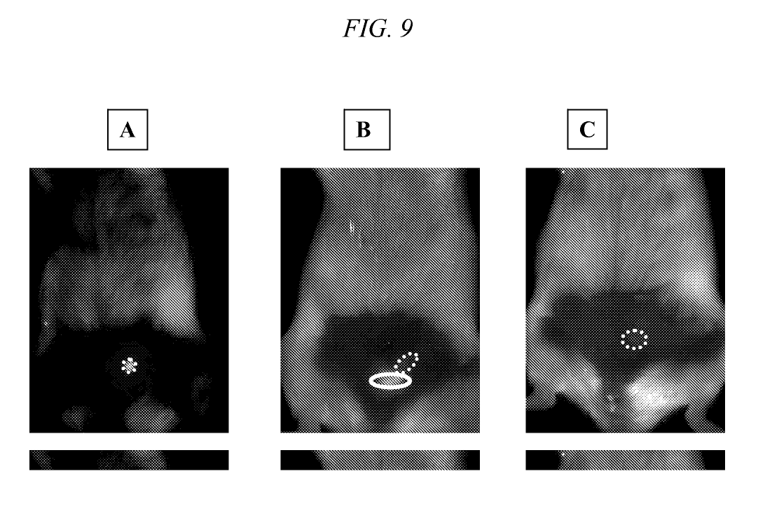

10⁴ 4T1 tumor cells marked with a red fluorescent protein were injected sub-cutaneously into mammary pad BALB/c female mice this constitutes Day O in FIG. 8a. At day 7 when the tumor reached an appropriate mass an shRNA comprising SEQ ID NO: 4, a complementary second strand, a binding moiety and a green fluorescent tag were injected into the mouse pad. In FIGS. 8 and 9, the tumor site is outlined approximately by shapes having dashed lines while the shRNA is represented outlined approximately by shapes having solid lines. In vivo imaging 24 hour later, FIG. 8b, shows the tumor as evinced by the red fluorescent tag and the shRNA localized proximal to the tumor site as evinced by the green fluorescent tag. At day 14, FIG. 8c, there is a reduction in tumor mass when compared to an untreated tumor. The experiment was repeated with the variation that the shRNA was injected when at a reduced tumor mass, day 4, and imaged 24 hours later, FIG. 9b. At day 14, a reduction in tumor mass was observed, FIG. 9c, when compared to an untreated tumor.

### Prophetic Example 5

The following is a prophetic protocol for siRNA gene therapy utilizing the compositions disclosed herein. Briefly, liposomes consisting of DOTAP and Cholesterol (1:1 molar ratio) will be prepared by thin film hydration then membrane extrusion to get 80-100 nm particle size. The particle size will be measured by using N4 PLUS Coulter particle size scattering instrument. Liposome nanoparticles will contain DOTAP/Cholesterol, protamine sulfate and the Hsp targeting siRNA oligonucleotides of the type disclosed in SEQ ID Nos. 4-6. To prepare 1 mg/kg bodyweight siRNA formulations, 200 μl liposome nano particles contains 13.5 μl siRNA, 10 μl (20 μg) protamine sulfate, 40 μl DOTAP and Cholesterol (1:1 molar ratio), 15 μl Transferrin (300 μg), 121.5 μl RNase free water. DOTAP, Cholesterol is commercially available from Avanti Polar Lipids, Inc., human transferrin in the iron-saturated, heat inactivated form is commercially available from BD Biosciences, and protamine sulfate Grade X isolated from salmon is commercially available from Sigma-Aldrich. The nanoparticle complex will be prepared by mixing the protamine sulfate, RNase free water, siRNA and allowed to stand at room temperature for 10 min before the addition of DOTAP/Cholesterol liposome, Transferrin complex. The liposome nanoparticles will be incubated at room temperature for 10 min before injection into animals.

10⁴ 4T1 tumor cells marked with a red fluorescent protein will be injected sub-cutaneously into mammary pad BALB/c female mice. siRNA treatment will begin when tumors attains the size of (20-30 mm²). siRNA formulations at a dose of 1-2 mg/kg (one injection per day for 3 days/week for 2-4 weeks) body weight will be injected into mice subcutaneously, i.v. or intra tumorally. The tumor regression will be monitored by in vivo imaging and tumor measurement by using digital caliper. During the course of treatment, tissues will be collected for siRNA distribution study and blood will be collected for cytokine measurement (in vivo toxicity) study. The results of these studies will be used in part to assess the ability of the Hsp compositions to reduce mammalian tumors, to decrease the metastatic potential of the tumors, and to evaluate the cross reactivity of differing mammalian sequences.

While various embodiments have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present disclosure. The discussion of a reference in the disclosure is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. An isolated double stranded ribonucleic acid (dsRNA) molecule that inhibits the expression of a target gene, the dsRNA comprising two strands of nucleotides wherein a first strand has a length of from 19 to 28 consecutive nucleotides and is substantially identical to a sequence in the target gene and wherein a second strand is substantially complementary to the first strand, and a binding moiety that binds a 3′ end of the first strand to a 5′ end of the second strand.

2. The dsRNA of claim 1 wherein the binding moiety comprises a polynucleotide linker.

3. The dsRNA of claim 2 wherein the polynucleotide linker is from 5 to 12 base pairs in length.

4. The dsRNA of claim 1 wherein the target gene encodes for a heat shock protein.

5. The dsRNA of claim 1 wherein the target gene comprises SEQ ID No: 3.

6. The dsRNA of claim 1 wherein the first strand comprises SEQ ID Nos: 4, 5, or 6.

7. The dsRNA of claim 1 wherein the first strand, the second strand, or both further comprise a marker protein.

8. The dsRNA of claim 7 wherein the marker protein is a fluorescent protein.

9. A vector family for the transduction of cells comprising the dsRNA of claim 1.

10. The vector family of claim 9 wherein the vector is a retroviral vector.

11. The vector family of claim 10 wherein the vector is a lentiviral vector.

12. The vector family of claim 9 further comprising promoters, ribosome binding sites, enhancer sequences, response elements, inducible elements, selectable markers, regulatory elements, or combinations thereof.

13. The vector family of claim 12 wherein the promoters comprise mouse UG RNA promoters, synthetic human H1RNA promoters, SV40 promoter, CMV promoters, RSV promoters, RNA polymerase II promoters, RNA polymerase III promoters, derivatives thereof, or combinations thereof.

14. A cell line comprising the dsRNA of claim 1.

15. The cell line of claim 14 wherein the cell line is a packaging cell line.

16. A non-human animal comprising the dsRNA of claim 1.

17. A method of treating an organism experiencing a proliferative disorder comprising administering a therapeutic amount of a composition comprising the dsRNA of claim 1.

18. The method of claim 17 wherein the proliferative disorder is evinced by tumor growth.

19. The method of claim 18 wherein the tumor growth is inhibited by from about 10% to about 95%.

20. The method of claim 18 wherein the metastatic potential of the tumor is reduced by from about 10% to about 95%.

21. A pharmaceutical composition comprising the dsRNA of claim 1 and an excipient.

22. The pharmaceutical composition of claim 21 further comprising a delivery system and a tumor targeting moiety.

23. The pharmaceutical composition of claim 22 wherein the delivery system comprises a liposome.

24. The pharmaceutical composition of claim 22 wherein the tumor targeting moiety comprises an antibody, transferrin, or combinations thereof.

25. An isolated double stranded ribonucleic acid molecule comprising a first strand of nucleotides that is substantially identical to SEQ ID NO:3 and a second strand that is substantially complementary to the first.

26. An isolated double stranded ribonucleic acid that inhibits expression of a protein encoded by a nucleic acid molecule comprising a sequence set forth in SEQ ID NO:3; wherein a first strand of the dsRNA is substantially identical to SEQ ID NO:3 and a second strand is substantially complementary to the first.

27. A vector family for the transduction of cells comprising the dsRNA of claim 26.

28. A pharmaceutical composition for reducing tumor growth and/or metastatic potential comprising the dsRNA of claim 26 and an excipient.
